(19) United States
(12) Patent Application Publication
(10) Pub. No.: US 2026/0127853 A1
(43) Pub. Date: May 7, 2026

(54) METHODS AND SYSTEMS FOR CHEMISTRY-AWARE AUTOMATED CLASSIFICATION OF AND INIHIBITATION PROTOCOLS FOR CORROSION AND MATERIAL DEGRADATION

(71) Applicant: Toyota Research Institute, Inc., Los Altos, CA (US)

(72) Inventors: Steven Bartholomew Joseph Torrisi, San Mateo, CA (US); Joseph Harold Montoya, Berkeley, CA (US)

(73) Assignees: Toyota Research Institute, Inc., Los Altos, CA (US); Toyota Jidosha Kabushiki Kaisha, Toyota-shi Aichi-ken (JP)

(21) Appl. No.: 18/938,868

(22) Filed: Nov. 6, 2024

(51) Int. Cl. G06V 10/764; G06V 10/56; G06V 10/60; G06V 10/77; G06V 10/774; G16C 20/70; G16C 60/00

(52) U.S. Cl. CPC G06V 10/764; G06V 10/56; G06V 10/60; G06V 10/7715; G06V 10/774; G16C 20/70; G16C 60/00

(57) ABSTRACT

An automated corrosion prediction system includes a processor and a memory communicably coupled to the processor and storing machine-readable instructions. The machine-readable instructions, when executed by the processor, cause the processor to train a multimodal machine learning (ML) model with data from a plurality of corrosion-related databases, execute an image analysis of an image of a corrosion product on a substrate and identify features of the image, map the image analysis to a description of the identified features, provide the description to the trained multimodal ML model, and automatically predict, within 60 seconds from receiving the description, at least one of a type and a chemistry of the corrosion product from the description using the trained multimodal ML model.

### TECHNICAL FIELD

The present disclosure relates generally to corrosion detection and corrosion remediation.

### BACKGROUND

Accurately identifying corrosion products and modes of corrosion is of interest to scientists, engineers, and companies of products that can experience corrosion. Traditional methods for identifying and analyzing corrosion products and modes of corrosion rely on a visual inspection and possible corrosion product analysis by trained experts, which can be time and cost intensive. Accordingly, systems and/or methods that provide enhanced prediction of corrosion products and modes of corrosion would be desirable.

The present disclosure addresses issues related to the identification of corrosion products, modes of corrosion and/or rates of corrosion, and other issues related to corrosion.

### SUMMARY

This section provides a general summary of the disclosure and is not a comprehensive disclosure of its full scope or all of its features.

In one form of the present disclosure, an automated corrosion prediction system includes a processor and a memory communicably coupled to the processor and storing machine-readable instructions that, when executed by the processor, cause the processor to train a multimodal machine learning (ML) model with data from a plurality of corrosion-related databases, execute an image analysis of an image of a corrosion product on a substrate and identify features of the image, map the image analysis to a description of the identified features, provide the description to the trained multimodal ML model, and automatically predict, within 60 seconds from receiving the description, at least one of a type and a chemistry of the corrosion product from the description using the trained multimodal ML model.

In another form of the present disclosure, an automated corrosion prediction system includes a digital camera and a processor and a memory communicably coupled to the processor and storing machine-readable instructions that, when executed by the processor, cause the processor to capture a digital image of a corrosion product on a substrate, execute an image analysis of the digital image and identify features of the image, map the image analysis to a description of the identified features, and predict at least one of a type and a chemistry of the corrosion product from the description with a multimodal machine learning (ML) model trained with at least two corrosion-related databases.

In still another form of the present disclosure, a method for automatically identifying corrosion includes executing an image analysis of a digital image of a corrosion product on a substrate and identify features of the digital image, mapping the image analysis to a description of the identified features, and predicting at least one of a type of corrosion and a chemistry of the corrosion product from the description with a multimodal machine learning (ML) model trained with at least two corrosion-related databases.

Further areas of applicability and various methods of enhancing the above technology will become apparent from the description provided herein. The description and specific examples in this summary are intended for purposes of illustration only and are not intended to limit the scope of the present disclosure.

### BRIEF DESCRIPTION OF THE DRAWINGS

The present teachings will become more fully understood from the detailed description and the accompanying drawings, wherein:

FIG. 1 illustrates a system for predicting a type of corrosion and/or a chemistry of a corrosion product according to one form of the present disclosure;

FIG. 2 is a block diagram for a system for predicting a type of corrosion and/or a chemistry of a corrosion product according to the teachings of the present disclosure;

FIG. 3 is a flow chart for a method of predicting a type of corrosion and/or a chemistry of a corrosion product according to the teachings of the present disclosure;

FIG. 4 is a flow chart for a method of predicting a type of corrosion and/or a chemistry of a corrosion product according to the teachings of the present disclosure;

FIG. 5 is a flow chart for a method of predicting a type of corrosion and/or a chemistry of a corrosion product according to the teachings of the present disclosure;

FIG. 6 is a flow chart for a method of predicting a type of corrosion and/or a chemistry of a corrosion product according to the teachings of the present disclosure;

FIG. 7 illustrates suspected corrosion on a vehicle fender;

FIG. 8 illustrates suspected corrosion near a battery terminal; and

FIG. 9 illustrates suspected corrosion on a vehicle frame member.

### DETAILED DESCRIPTION

The present disclosure provides systems and methods for predicting a type of corrosion (also referred to herein as “corrosion type”) and/or a chemistry of a corrosion product for corrosion on a substrate. As used herein, the term “corrosion” refers to the degradation of a metal and/or alloy due to a reaction of the metal and/or alloy with its surrounding environment. Stated differently, corrosion is the gradual degradation and deterioration of a material via chemical or electrochemical reaction with an environment in contact with the material. In some variations, the systems and methods further provide a corrosion inhibition protocol after a corrosion type and/or corrosion product chemistry has been predicted. As used herein, the phrase “corrosion inhibition protocol” refers to an instruction to be performed or executed by an individual in an effort to reduce corrosion and/or prevent corrosion from occurring.

The systems according to the teachings of the present disclosure perform or execute image analysis of an image (e.g., a digital image) of a corrosion product and identify or extract features of the image, which are then mapped onto a description of the identified features. Then a multimodal machine learning (ML) model, trained with data from a plurality corrosion-related databases, evaluates the description of the identified features and predicts a type of corrosion the substrate is experiencing and/or a chemistry of the corrosion product. In some variations, the systems provide a corrosion inhibition protocol for the predicted type of corrosion and/or chemistry of the corrosion product. For example, in at least one variation the corrosion inhibition protocol is provided by the same multimodal ML model or a different multimodal ML model having been trained with a plurality corrosion inhibition databases.

It should be understood that traditional identification of a type of corrosion and/or a corrosion product includes a visual inspection of a corrosion product by a trained individual (e.g., a corrosion engineer). Also, traditional identification may further include analysis of the corrosion product (e.g., an oxide scale, an oxide powder, etc.) using analytical techniques such as x-ray diffraction (XRD), scanning electron microscopy (SEM), energy dispersive spectroscopy (EDS), among others. Examples of corrosion types that can be identified include galvanic corrosion, crevice corrosion, pitting corrosion, surface or uniform corrosion (e.g., “rust”), filiform corrosion, intergranular corrosion, and deposit corrosion, among others. In addition, morphologies o such corrosion and/or corrosion products include an average grain size of a corrosion product, an average grain size aspect ratio of a corrosion product, a surface roughness of the corrosion product, flakes of a corrosion product, pits in a substrate, pits in a coating, cracks in a corrosion product, cracks in a coating, ridges in a corrosion product, and ridges in a coating, among others.

Accordingly, the systems and methods according to the teachings of the present disclosure improve, in terms of cost and time, current techniques, systems and/or methods for the identification of corrosion types, corrosion products and/or corrosion inhibition protocols.

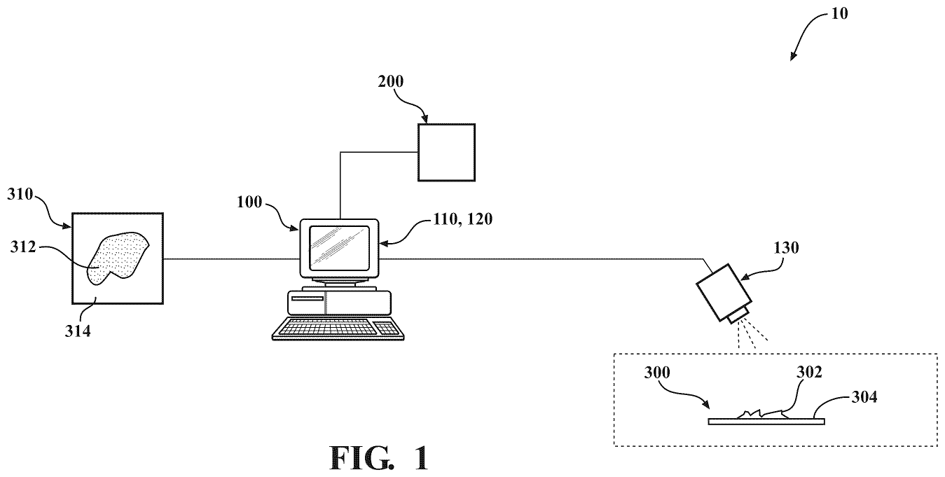

Referring to FIG. 1, a system 10 for predicting a corrosion type and/or a chemistry of a corrosion product on a substrate according to one form of the present disclosure is shown. The system 10, and other systems disclosed herein, includes multiple elements. It should be understood that in some variations the system 10 may not necessarily include all of the elements shown in FIG. 1 and the system 10 can have any combination of the various elements shown in FIG. 1. Further, the system 10 can include other elements in addition to those shown in FIG. 1. In some variations, the system 10 is implemented without one or more of the elements shown in FIG. 1. Also, and while FIG. 1 illustrates the various elements located at various positions within the system 10, it should be understood that one or more of these elements can be located external to the system 10. Further, the elements shown may be physically separated by large distances.

The elements of the disclosed system 10 are shown in FIG. 1 and will be described along with subsequent figures. For simplicity and clarity of illustration, where appropriate, reference numerals have been repeated among the different figures to indicate corresponding or analogous elements. In addition, while the discussion outlines numerous specific details to provide a thorough understanding of the system 10 described herein, those of ordinary skill in the art will understand that the variations or forms described herein may be practiced using various combinations of these elements.

The system 10 includes a processor 110 and a memory 120, e.g., as part of a computer 100. Also, the processor 110 is in communication with and/or has been in communication with a plurality of corrosion-related databases 200 and the memory 120 includes at least one digital image 310 of a corrosion product 312. In some variations, the digital image 310 includes a substrate 314 on which the corrosion product 312 has formed.

In at least one variation, the system 10 includes a digital camera 130 configured to take or capture the at least one digital image 310 of corrosion 300 with a corrosion product 302 that has formed on a substrate 304. In the alternative, or in addition to, the corrosion 300 includes a deteriorated substrate 304 without the presence of a corrosion product, e.g., a substrate 304 of a pitted surface due to pitting corrosion in a liquid containing environment. It should be understood that the digital camera 130 is in communication (wireless or wired) with the processor 110 and memory 120 and the captured digital image 310 is stored in the memory 120.

In other variations, the system 10 does not include the digital camera 130 and the at least one digital image 310 is provided from a different source, e.g., from a flash drive, email, and the like. And in at least one variation, the system 10 includes, i.e., the memory 120 stores, at least one digital image 310 captured with the digital camera 130 and at least one digital image 310 provided by a different source, e.g., from a flash drive, email, and the like.

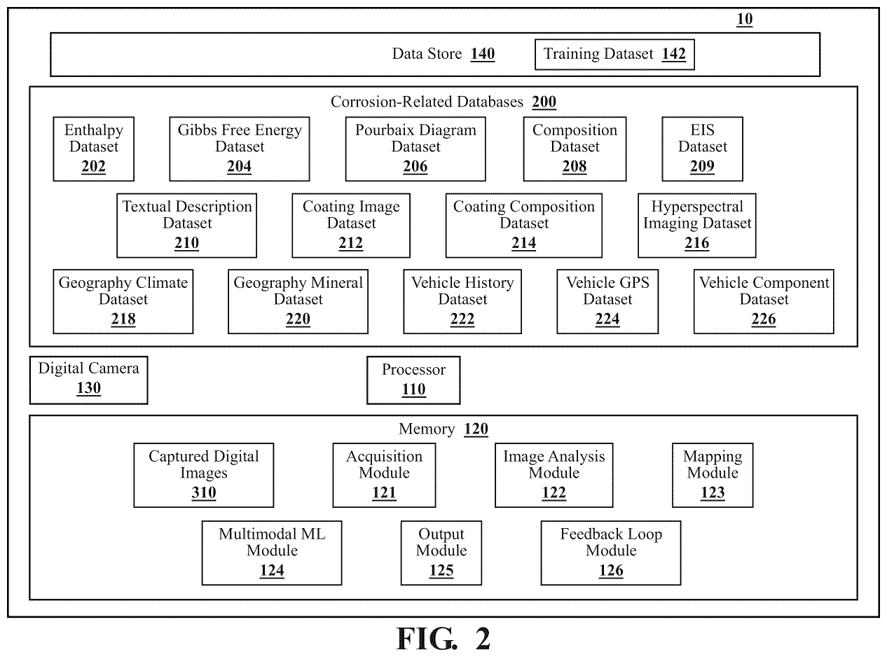

Referring to FIG. 2, a block diagram of the system 10 or the system 20 (referred to herein simply as “system 10”) is shown. The system 10 includes the processor 110, the memory 120, and the plurality of corrosion-related databases 200. In some variations, the system 10 includes the digital camera 120 and may or may not include a data store 140.

The processor 110 is in communication with the memory 120, and the memory 120 can include the one or more captured digital images 310, as well as an acquisition module 121, an image analysis module 122, a mapping module 123, a multimodal ML module 124, an output module 125 and/or a feedback loop module 126 (collectively referred to herein as “modules 121-126”). The modules 121-126 are, for example, computer-readable instructions that when executed by the processor 110 cause the processor 110 to perform the various functions disclosed herein.

The acquisition module 121 can include instructions that function to control the processor 100 to select a training dataset 142 stored in a data store 140. In some variations, the training dataset 142 is a multimodal training dataset, i.e., the training dataset 142 includes data from a plurality of different databases. And in at least one variation, the training dataset 142 includes data from two or more, e.g., three or more, or four or more, of the plurality of datasets within the plurality of corrosion-related databases 200.

The image analysis module 122 can include instructions that function to control the processor 100 to execute one or more of image enhancement, image restoration, image segmentation, image representation and description, and image analysis on a captured digital image. As used herein, the phrase “image enhancement” refers to enhancing the visual quality of an image, e.g., by increasing contrast, reducing noise, and removing artifacts, and the phrase “image restoration” refers to removing degradation such as a blurring, noise, and distortion form an image. As used herein, the phrase “image segmentation” refers to dividing an image into regions or segments such that each region or segment corresponds to a specific object or feature in the image, and the phrase “image representation and description” refers to representing or transforming an image into a format that is communicable (i.e., can be read by) to the mapping module 123 and/or the multimodal ML module 124. And as used herein, the phrase “image analysis” refers to extracting information (e.g., recognizing objects, detecting patterns, quantifying patterns, etc.) from an image via an algorithm and/or mathematical model.

The mapping module 123 can include instructions to map an image analysis to a description (e.g., a text description) that is communicable with the multimodal ML module 124. For example, in some variations mapping the image analysis executed by the processor 100 includes or incorporates prior knowledge of a substrate or part from which an image of a corrosion product is captured. And in such variations, the prior knowledge provides context or aids in interpreting or mapping the image analysis by the mapping module 123. For example, the mapping module 123 can include prior knowledge of colors associate with different corrosion products such as the color “green” associate with corrosion of copper and the color “brown” or “reddish brown” associated the corrosion of iron, and such prior knowledge thereby aids or enhances the mapping of image analysis to a description which is subsequently provided to the multimodal ML module 124.

The multimodal ML module 124 can include instructions to predict a type and/or a chemistry of a corrosion product for which a digital image has been captured. Not being bound by theory, the multimodal ML module 124 includes instructions, that when executed by the processor 110, results in representation, alignment, reasoning, generation, and transference as disclosed in the references titled “Foundations & Trends in Multimodal Machine Learning: Principles, Challenges, and Open Questions”, by Laing et al., ACM Comput. Surv., Vol. 56, No. 10, Article 264, June 2024, and “An Introduction to Vision-Language Modeling” by Boardes et al., arXiv:2405.17247v1 [cs.LG], 27 May 2024, both of which are incorporated herein by reference. Accordingly, the image analysis module 122 processes a digital image 310 as described above, and the mapping module 123 distills the processed digital image into a description with optional prior knowledge information, and the multimodal ML module used the digital image 310, processed digital image, description, and/or optional prior knowledge information to predict a type and/or a chemistry of the corrosion product for which the digital image was captured.

In some variations, the multimodal ML module 124 is trained with data from two or more corrosion-related datasets within the corrosion-related databases 200. In at least one variation, the multimodal ML module 124 predicts the type and/or chemistry of the corrosion product as a function of a comparison of the description of the captured digital image and data from one or more of the corrosion-related datasets within the corrosion-related databases 200. For example, in some variations the multimodal ML module 124 determines/compares if a textual description of a captured image matches to a level of certainty a textual description in one or more of the corrosion-related datasets within the corrosion-related databases 200. In other variations, the multimodal ML module 124 determines/compares if a set of features extracted a captured image matches using the image analysis module 122 match to a level of certainty a set of features in one or more of the corrosion-related datasets within the corrosion-related databases 200.

The output module 125 can include instructions to output information related to one or more of a prediction of a type of corrosion for which a digital image has been captured, a prediction of a chemistry of a corrosion product for which a digital image has been captured, a certainty score for a predicted type of corrosion for which a digital image has been captured, and a certainty score for a predicted corrosion product for which a digital image has been captured. In some variations, the output module 125 outputs the above noted information to a user interface of a computer.

The feedback loop module 126 can include instructions to provide one or more of a prediction of a type of corrosion for which a digital image has been captured, a prediction of a chemistry of a corrosion product for which a digital image has been captured, a certainty score for a predicted type of corrosion for which a digital image has been captured, and a certainty score for a predicted corrosion product for which a digital image has been captured to the training dataset 142 and/or to one or more of the corrosion-related datasets within the corrosion-related databases 200.

The data store 140 stores, among other things, a training dataset 142, as will be discussed further below. The data store 140, in one embodiment, is constructed as an electronic data structure stored in the memory 120 or another data store, such as a cloud-based storage, a removable memory device, or another suitable location that is accessible to images and modules stored in the memory 120. The data store 140 is configured with routines that can be executed by the processor 110 for analyzing stored data, providing stored data, organizing stored data, and so on. Thus, in some variations, the data store 140 stores data described above (as well as other data) used by modules stored in the memory 120 in executing various functions.

The corrosion-related databases 200 includes a plurality of corrosion-related datasets that can be used to training a multimodal ML module and/or be used for a comparison of data contained therein with results from an image analysis module, mapping module, multimodal ML module 124. For example, examples of corrosion-related datasets include an enthalpy dataset 202, a Gibbs Free Energy dataset 204, a Pourbaix diagram dataset 206, chemical composition dataset 208 (shown as “Composition Dataset 208” in FIG. 2), a corrosion product Electrochemical Impedance Spectroscopy (EIS) dataset 209 (shown as “EIS Dataset 209” in FIG. 2), a textual description dataset 210, a coating image dataset 212, a coating composition dataset 214, a hyperspectral imaging dataset 216, a geography climate dataset 218, a geography mineral dataset 220, a vehicle age dataset 222, a vehicle global positioning system (GPS) dataset 224, and a vehicle component dataset 226 among others (collectively referred to herein as “datasets 202-226”). In some variations, all of the datasets 202-226 are stored in the memory 120, while in other variations one or more of the datasets 202-226 are not stored in the memory 120. For example, in some variations one or more of the datasets 202-226 are stored in a cloud-based storage memory, a removable memory device, or another suitable location that is accessible to the processor 110, the acquisition module 121, and/or the multimodal ML module 124.

The enthalpy dataset 202 can include enthalpy of formation for corrosion products, e.g., the enthalpy of formation for oxides and/or sulfides as found in the NIST Chemistry WebBook found at webbook.nist.gov/chemistry/ and/or Thermo-Calc Databases found at thermos.calc/products/databases (subscription required). And the Gibbs Free Energy dataset 204 can include Gibbs Free Energy of formation for corrosion products, e.g., the enthalpy of formation for oxides and/or sulfides also found in the NIST Chemistry WebBook and/or Thermo-Calc Databases. Ther Pourbaix diagram dataset 205 can include maps of conditions of equilibrium potential and acidity/basicity (i.e., pH) for stable chemical species of a material exposed to a liquid environment as found at next-gen.materialsproject.org and Thermo-Calc Databases. The chemical composition dataset 208 can include chemical compositions for known corrosion products as found at Thermo-Calc Databases. The textual description dataset 210 can include textual descriptions of known corrosion products as found in the CORR-DATA_Database.zip file available at the NIST Public Data Repository, and such data provides textual observations of samples in corrosive environments under particular conditions.

The coating image dataset 212 can include digital images of different coatings, including but not limited to paint coatings, polymer coatings, and metallic coatings. In some variations, the coating image dataset can include digital images of different coatings, on a substrate, and with degradation present. For example, coatings, on a substrate, and with a scratch thereon and/or therein, coatings, on a substrate, with a hole or aperture therein, coatings, on substrate, with a corrosion product extending through the coating and visible to the naked eye, and coatings, on substrate, with a corrosion product between the coating and the substrate and not visible to the naked eye, among others. In addition, in some variations the coating image dataset 212 may or may not include digital images that have been subjected to image analysis. And in such variations, the coating image dataset 212 may contain image one or more of image enhancement data, image restoration data, image segmentation data, image representation and description data, and image analysis data of the digital images. In the alternative, or in addition to, one or more of image enhancement data, image restoration data, image segmentation data, image representation and description data, and image analysis data of the digital images can be stored in a different dataset.

The coating composition dataset 214 can include chemical compositions of different coatings, including but not limited to chemical compositions of paint coatings, polymer coatings, and metallic coatings, among others. The hyperspectral imaging dataset 216 can include hyperspectral imaging data on known corrosion products. The geography climate dataset 218 includes historical weather data for geographic locations in a given country, state, and/or city, for example as found at weather.gov/dtx/WeatherHistory. And the geography mineral dataset 220 can include data on minerals as a function of geography, for example as found at mindat.org. The vehicle history dataset 222 can includes data on the age and/or service history of a plurality of vehicles, the vehicle GPS dataset 224 can include GPS data on a plurality of vehicles and/or service location history on a plurality of vehicles, and the vehicle component dataset 226 can include the history of one or more components for a plurality of vehicles. For example, in some variations the vehicle history dataset 220 includes a plurality of Vehicle Identification Numbers (VINs) selectively tagged with a year of manufacture, a location of assembly, and/or service history data, the vehicle GPS dataset 224 includes a plurality of VINs selectively tagged with GPS data such that location history of the plurality of vehicles is available. And in at least one variation, the vehicle component dataset 226 includes a plurality of VINs selectively tagged with a location of manufacture, shipment route(s) between a location of manufacture and a location of vehicle assembly, method(s) of shipment between a location of manufacture and a location of vehicle assembly, and date(s) of shipment from a location of manufacture and a location of vehicle assembly.

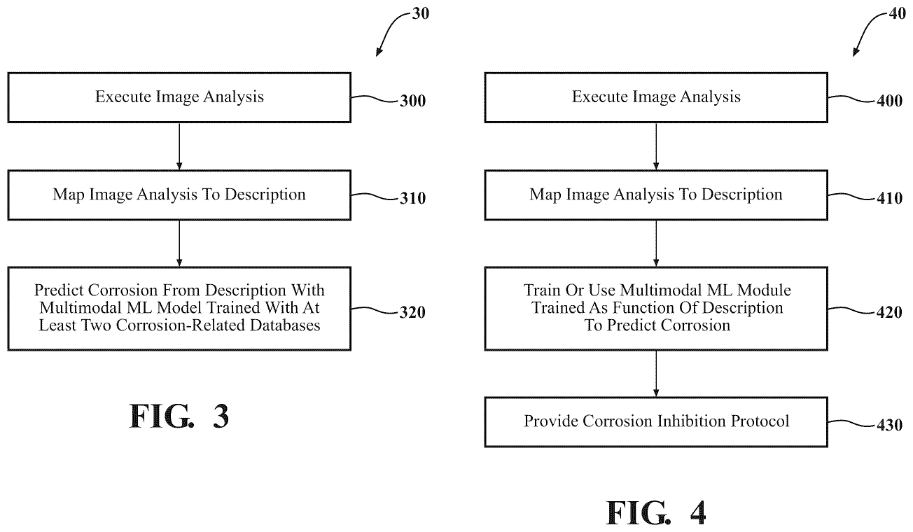

Referring to FIG. 3, and with reference to FIG. 2, a flow chart for a method 30 according to one form of the present disclosure is shown. The method 30 includes executing an image analysis on a captured digital image 310 using the image analysis module 122 at 300. The executed image analysis is mapped to a description using the mapping module 123 at 310 and the multimodal ML module 124, having been trained with at least two corrosion-related databases and having received the description, predicts a corrosion type and/or a corrosion product at 320.

Referring to FIG. 4, and with reference to FIG. 2, a flow chart for a method 40 according to another form of the present disclosure is shown. The method 40 includes executing an image analysis on a captured digital image 310 using the image analysis module 122 at 400 and mapping the executed image analysis to a description using the mapping module 123 at 410. In some variations, the multimodal ML module 124 is trained with at least two of the datasets 202-226 and then predicts a corrosion type and/or corrosion product at 420, while in other variations a multimodal ML module 124 is already trained with at least two of the datasets 202-226 and predicts a corrosion type and/or corrosion product at 420. The method 50 also, and optionally, includes providing a corrosion inhibition protocol at 430.

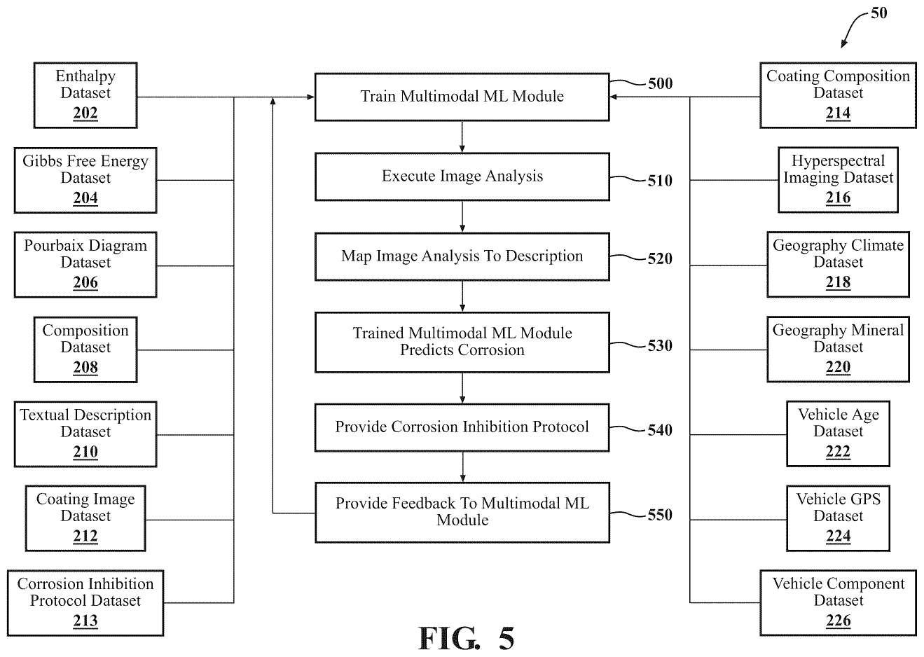

Referring to FIG. 5, and with reference to FIG. 2, a flow chart for a method 50 according to another form of the present disclosure is shown. The method includes training the multimodal ML module 124 with at least two of the datasets 202-226 at 500, executing an image analysis on a captured digital image 310 using the image analysis module 122 at 510 and mapping the executed image analysis to a description using the mapping module 123 at 520. The description is provided to the trained multimodal ML module 124 and the trained multimodal module 124 predicts a corrosion type and/or corrosion product at 530. In some variations, the trained multimodal module 124 provides a corrosion inhibition protocol at 540, and in at least one variation the method 50 includes providing the predicted corrosion type, corrosion product, and/or corrosion inhibition protocol to the multimodal ML module 124 at 550.

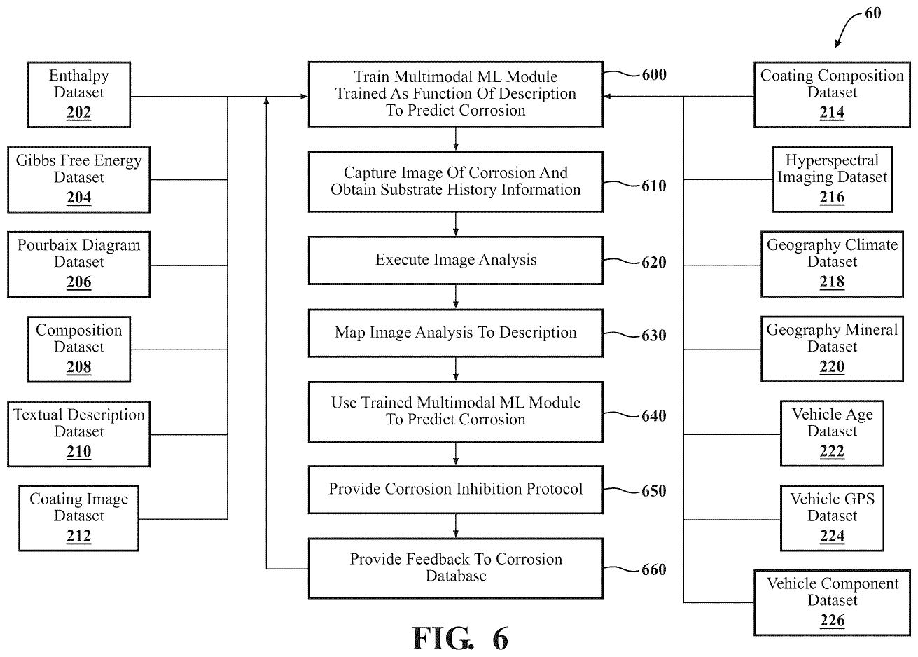

Referring to FIG. 6, and with reference to FIG. 2, a flow chart for a method 60 according to another form of the present disclosure is shown. The method includes training the multimodal ML module 124 with at least two of the datasets 202-226 at 600 and capturing an image of corrosion at 610. In some variations, the method 60 also includes obtaining a history of the substrate where the corrosion is present at 610. The method 60 executes an image analysis on a captured digital image 310 using the image analysis module 122 at 620 and maps the executed image analysis to a description using the mapping module 123 at 630. In variations where the history of the substrate where the corrosion is present is obtained, the description can include the history of the substrate. The description is provided to the trained multimodal ML module 124 and the trained multimodal module 124 predicts a corrosion type and/or corrosion product at 640. In some variations, the trained multimodal module 124 provides a corrosion inhibition protocol at 650, and in at least one variation the method 60 includes providing the predicted corrosion type, corrosion product, and/or corrosion inhibition protocol to the multimodal ML module 124 at 660.

In an effort to better describe the systems and methods disclosed herein, and yet no limit the scope thereof, examples of using the system 10 are provided below.

### Example 1

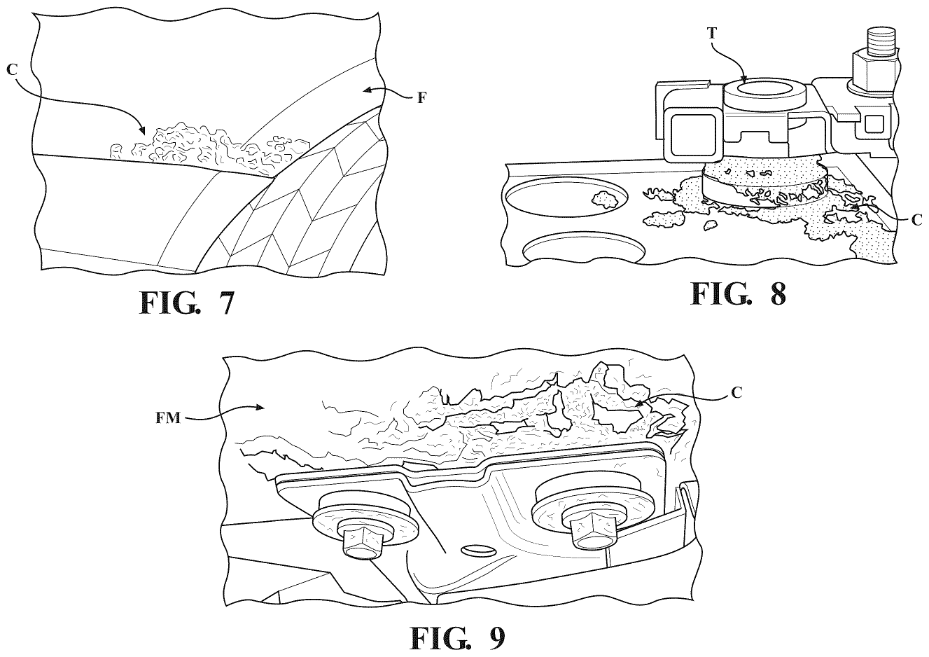

Referring to FIG. 1, the memory 120 includes machine-readable instructions that cause the processor 110 to train the multimodal ML module 124 with truth data in the Training Dataset 142 from the Gibbs Free Energy Dataset 204, Pourbaix Diagram Dataset 206, Textual Description Dataset 210, Coating Image Dataset 212, Geography Climate Dataset 218, Vehicle History Dataset 222, Vehicle GPS Dataset 222, and Vehicle Component Dataset 222. In addition, a technician at a vehicle repair center captures an image (with digital camera 130) of suspected corrosion ‘C’ on a vehicle fender ‘F’ as illustrated in FIG. 7.

The image is transmitted to the Captured Digital Images 310 and the Acquisition Module 121 provides the image to the Image Analysis Module 122. In addition, the captured image is tagged with the vehicle VIN number and vehicle service location data. The Image Analysis Module 122 executes an image analysis and identifies features of the image, the Acquisition Module 121 provides the image analysis to the Mapping Module 123, the Mapping Module maps the image analysis to a description of the identified features, and the Acquisition Module 121 provides the description of the identified features to the multimodal ML module 124. The multimodal ML module 124, based on being trained with the truth data, predicts and the Output Module 125 provides a conclusion that the suspected corrosion is corrosion of the fender due to exposure of salt from the ocean. That is, the corrosion of the fender is a function of the vehicle being owned and operated near an ocean in a warmer climates (e.g., Tampa, Florida) and not due to road salt corrosion experienced in colder climates (e.g., Detroit, Michigan). And in some variations, the multimodal ML module 124 predicts and the Output Module 125 provides a remedy for reducing or inhibiting continued corrosion of the fender F.

### Example 2

Still referring to FIG. 1, the memory 120 includes machine-readable instructions that cause the processor 110 to train the multimodal ML module 124 with truth data in the Training Dataset 142 from the Gibbs Free Energy Dataset 204, Pourbaix Diagram Dataset 206, Textual Description Dataset 210, Coating Image Dataset 212, Geography Climate Dataset 218, Vehicle History Dataset 222, Vehicle GPS Dataset 222, and Vehicle Component Dataset 222. In addition, a technician at a vehicle repair center captures an image (with digital camera 130) of suspected corrosion ‘C’ near a battery terminal ‘T’ as illustrated in FIG. 8.

The image is transmitted to the Captured Digital Images 310 and the Acquisition Module 121 provides the image to the Image Analysis Module 122. In addition, the captured image is tagged with the vehicle VIN number. The Image Analysis Module 122 executes an image analysis and identifies features of the image, the Acquisition Module 121 provides the image analysis to the Mapping Module 123, the Mapping Module maps the image analysis to a description of the identified features, and the Acquisition Module 121 provides the description of the identified features to the multimodal ML module 124. The multimodal ML module 124, based on being trained with the truth data, predicts and the Output Module 125 provides a conclusion that the suspected corrosion is battery corrosion due to the age of the battery and/or a battery leak. That is, the corrosion a function of the age of the battery or the battery having a leak. And in some variations, the multimodal ML module 124 predicts and the Output Module 125 provides a remedy for reducing or inhibiting the corrosion C, i.e., to replace the battery.

### Example 3

Still referring to FIG. 1, the memory 120 includes machine-readable instructions that cause the processor 110 to train the multimodal ML module 124 with truth data in the Training Dataset 142 from the Gibbs Free Energy Dataset 204, Pourbaix Diagram Dataset 206, Textual Description Dataset 210, Coating Image Dataset 212, Geography Climate Dataset 218, Vehicle History Dataset 222, Vehicle GPS Dataset 222, and Vehicle Component Dataset 222. In addition, a technician at a vehicle repair center captures an image (with digital camera 130) of suspected corrosion ‘C’ on a vehicle frame member ‘FM’ as illustrated in FIG. 9.

The image is transmitted to the Captured Digital Images 310 and the Acquisition Module 121 provides the image to the Image Analysis Module 122. In addition, the captured image is tagged with the vehicle VIN number. The Image Analysis Module 122 executes an image analysis and identifies features of the image, the Acquisition Module 121 provides the image analysis to the Mapping Module 123, the Mapping Module maps the image analysis to a description of the identified features, and the Acquisition Module 121 provides the description of the identified features to the multimodal ML module 124. The multimodal ML module 124, based on being trained with the truth data, predicts and the Output Module 125 provides a conclusion that the suspected corrosion is corrosion of the frame member FM due to exposure to road salt. That is, the corrosion of the frame member FM is a function of the vehicle being owned and operated in colder climates where roads are salted during the winter (e.g., Detroit, Michigan) and not due to salt from the ocean and warmer climates (e.g., Tampa, Florida). And in some variations, the multimodal ML module 124 predicts and the Output Module 125 provides a remedy for reducing or inhibiting continued corrosion of the frame member FM.

In some variations, the multimodal ML module 124 predicts and the Output Module 125 provides the conclusion noted above in less than 60 seconds, e.g., less than 30 seconds, without examination of the corrosion ‘C’ by a corrosion expert, after the image is transmitted to the Captured Digital Images 310. And in at least one variation, the multimodal ML module 124 predicts and the Output Module 125 provides the conclusion noted above in less than 15 seconds, without examination of the corrosion ‘C’ by a corrosion expert, after the image is transmitted to the Captured Digital Images 310. Stated differently, it should be understood that the time required a corrosion expert to evaluate an image and predict at least one of a type and a chemistry of a corrosion product from the image as a function of data from two or more of a corrosion product enthalpy database, a corrosion product Gibbs free energy database, a corrosion product chemical composition database, a corrosion product Electrochemical Impedance Spectroscopy database, a Pourbaix diagram database, a corrosion product textual description database, a coatings image database, a coatings chemical composition database, a corrosion product hyperspectral image database, a geography climate database, a geography mineral database, a vehicle age database, and a vehicle GPS database is much greater (longer) than 60 seconds, for example, hours if not days.

As used herein, the phrase at least one of A, B, and C should be construed to mean a logical (A or B or C), using a non-exclusive logical “or.” It should be understood that the various steps within a method may be executed in different order without altering the principles of the present disclosure. Disclosure of ranges includes disclosure of all ranges and subdivided ranges within the entire range.

The headings (such as “Background” and “Summary”) and sub-headings used herein are intended only for general organization of topics within the present disclosure and are not intended to limit the disclosure of the technology or any aspect thereof. The recitation of multiple variations or forms having stated features is not intended to exclude other variations or forms having additional features, or other variations or forms incorporating different combinations of the stated features.

As used herein the term “about” when related to numerical values herein refers to known commercial and/or experimental measurement variations or tolerances for the referenced quantity. In some variations, such known commercial and/or experimental measurement tolerances are +/−10% of the measured value, while in other variations such known commercial and/or experimental measurement tolerances are +/−5% of the measured value, while in still other variations such known commercial and/or experimental measurement tolerances are +/−2.5% of the measured value. And in at least one variation, such known commercial and/or experimental measurement tolerances are +/−1% of the measured value.

The flowcharts and block diagrams in the figures illustrate the architecture, functionality, and operation of possible implementations of systems, methods, and computer program products according to various embodiments. In this regard, a block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. Any kind of processing system or another apparatus adapted for carrying out the methods described herein is suited. A typical combination of hardware and software can be a processing system with computer-usable program code that, when being loaded and executed, controls the processing system such that it carries out the methods described herein. The systems, components and/or processes also can be embedded in a computer-readable storage, such as a computer program product or other data programs storage device, readable by a machine, tangibly embodying a program of instructions executable by the machine to perform methods and processes described herein. These elements also can be embedded in an application product which comprises the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk drive (HDD), a solid-state drive (SSD), a ROM, an EPROM or flash memory, a portable compact disc read-only memory (CD-ROM), a digital versatile disc (DVD), an optical storage device, a magnetic storage device, or any suitable combination of the foregoing. In the context of this document, a computer-readable storage medium may be any tangible medium that can contain or store a program for use by or in connection with an instruction execution system, apparatus, or device.

Generally, modules as used herein include routines, programs, objects, components, data structures, and so on that perform particular tasks or implement particular data types. In further aspects, a memory generally stores the noted modules. The memory associated with a module may be a buffer or cache embedded within a processor, a RAM, a ROM, a flash memory, or another suitable electronic storage medium. In still further aspects, a module as envisioned by the present disclosure is implemented as an ASIC, a hardware component of a system on a chip (SoC), as a programmable logic array (PLA), or as another suitable hardware component that is embedded with a defined configuration set (e.g., instructions) for performing the disclosed functions.

Program code embodied on a computer-readable medium may be transmitted using any appropriate medium, including but not limited to wireless, wireline, optical fiber, cable, radio frequency (RF), etc., or any suitable combination of the foregoing. Computer program code for carrying out operations for aspects of the present arrangements may be written in any combination of one or more programming languages, including an object-oriented programming language such as Java™, Smalltalk, C++, Python or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that a form or variation can or may comprise certain elements or features does not exclude other forms or variations of the present technology that do not contain those elements or features.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one variation, or various variations means that a particular feature, structure, or characteristic described in connection with a form or variation, or particular system is included in at least one variation or form. The appearances of the phrase “in one variation” (or variations thereof) are not necessarily referring to the same variation or form. It should be also understood that the various method steps discussed herein do not have to be carried out in the same order as depicted, and not each method step is required in each variation or form.

The foregoing description of the forms and variations has been provided for purposes of illustration and description. It is not intended to be exhaustive or to limit the disclosure. Individual elements or features of a particular form or variation are generally not limited to that particular form or variation, but, where applicable, are interchangeable and can be used in a selected form or variation, even if not specifically shown or described. The same may also be varied in many ways. Such variations should not be regarded as a departure from the disclosure, and all such modifications are intended to be included within the scope of the disclosure.

What is claimed is:

1. An automated corrosion prediction system comprising:
a processor and a memory communicably coupled to the processor and storing machine-readable instructions that, when executed by the processor, cause the processor to:
train a multimodal machine learning (ML) model with data from a plurality of corrosion-related databases;
execute an image analysis of an image of a corrosion product on a substrate and identify features of the image;
map the image analysis to a description of the identified features;
provide the description to the trained multimodal ML model; and
automatically predict, within 60 seconds from receiving the description, at least one of a type and a chemistry of the corrosion product from the description using the trained multimodal ML model.

2. The automated corrosion prediction system according to claim 1 further comprising a digital camera, wherein the machine-readable instructions that, when executed by the processor, cause the processor to capture a digital image of the corrosion product.

3. The automated corrosion prediction system according to claim 1, wherein the image analysis comprises extraction of one or more characteristic features selected from the group consisting of morphology of the corrosion product, color of the corrosion product, and fluorescent signals emitted by the corrosion product.

4. The automated corrosion prediction system according to claim 3, wherein the morphology of the corrosion product comprises one or more of an average grain size of the corrosion product, and an average grain size aspect ratio of the corrosion product, surface roughness of the corrosion product, flakes of a corrosion product, pits in a substrate, pits in a coating, cracks in a corrosion product, cracks in a coating, ridges in a corrosion product, and ridges in a coating.

5. The automated corrosion prediction system according to claim 1, wherein the at least two corrosion-related are selected from the group consisting of a corrosion product image database, a corrosion product enthalpy database, a corrosion product Gibbs free energy database, a corrosion product chemical composition database, a corrosion product Electrochemical Impedance Spectroscopy database, a Pourbaix diagram database, a corrosion product textual description database, a coatings image database, a coatings chemical composition database, a corrosion product hyperspectral image database, a geography climate database, a geography mineral database, a vehicle age database, and a vehicle GPS database.

6. The automated corrosion prediction system according to claim 5, wherein the at least two corrosion-related databases is at least three corrosion-related databases.

7. The automated corrosion prediction system according to claim 5, wherein the at least two corrosion-related databases is at least five corrosion-related databases.

8. The automated corrosion prediction system according to claim 1, wherein the description of the identified features is a textual description.

9. The automated corrosion prediction system according to claim 1, wherein the description of the identified features is a textual description and a feature description.

10. The automated corrosion prediction system according to claim 9, wherein the feature description is a vector description.

11. An automated corrosion prediction system comprising:
a digital camera;
a processor and a memory communicably coupled to the processor and storing machine-readable instructions that, when executed by the processor, cause the processor to:
capture a digital image of a corrosion product on a substrate;
execute an image analysis of the digital image and identify features of the image;
map the image analysis to a description of the identified features; and
predict at least one of a type and a chemistry of the corrosion product from the description with a multimodal machine learning (ML) model trained with at least two corrosion-related databases.

12. The automated corrosion prediction system according to claim 11, wherein the image analysis comprises extraction of one or more characteristic features selected from the group consisting of morphology of the corrosion product, color of the corrosion product, and fluorescent signals emitted by the corrosion product.

13. The automated corrosion prediction system according to claim 12, wherein the morphology of the corrosion product comprises one or more of an average grain size of the corrosion product, and an average grain size aspect ratio of the corrosion product, surface roughness of the corrosion product, flakes of a corrosion product, pits in a substrate, pits in a coating, cracks in a corrosion product, cracks in a coating, ridges in a corrosion product, and ridges in a coating.

14. The automated corrosion prediction system according to claim 11, wherein the at least two corrosion-related are selected from the group consisting of a corrosion product image database, a corrosion product enthalpy database, a corrosion product Gibbs free energy database, a corrosion product chemical composition database, a corrosion product Electrochemical Impedance Spectroscopy database, a Pourbaix diagram database, a corrosion product textual description database, a coatings image database, a coatings chemical composition database, a corrosion product hyperspectral image database, a geography climate database, a geography mineral database, a vehicle age database, and a vehicle GPS database.

15. The automated corrosion prediction system according to claim 11, wherein the description of the identified features is a textual description.

16. A method for automatically identifying corrosion, the method comprising:
executing an image analysis of a digital image of a corrosion product on a substrate and identify features of the digital image;
mapping the image analysis to a description of the identified features; and
predicting at least one of a type of corrosion and a chemistry of the corrosion product from the description with a multimodal machine learning (ML) model trained with at least two corrosion-related databases.

17. The method according to claim 16, wherein the identified features are one or more features selected from the group consisting of morphology of the corrosion product, color of the corrosion product, and fluorescent signals emitted by the corrosion product.

18. The method according to claim 17, wherein the morphology of the corrosion product comprises one or more of an average grain size of the corrosion product, and an average grain size aspect ratio of the corrosion product, surface roughness of the corrosion product, flakes of a corrosion product, pits in a substrate, pits in a coating, cracks in a corrosion product, cracks in a coating, ridges in a corrosion product, and ridges in a coating.

19. The method according to claim 16, wherein the description of the identified features is a textual description.

20. The method according to claim 16, wherein the at least two corrosion-related databases are selected from the group consisting of a corrosion product image database, a corrosion product enthalpy database, a corrosion product Gibbs free energy database, a corrosion product chemical composition database, a corrosion product Electrochemical Impedance Spectroscopy database, a Pourbaix diagram database, a corrosion product textual description database, a coatings image database, a coatings chemical composition database, a corrosion product hyperspectral image database, a geography climate database, a geography mineral database, a vehicle age database, and a vehicle GPS database.
